(19) United States
(12) Patent Application Publication
(10) Pub. No.: US 2024/0000343 A1
(43) Pub. Date: Jan. 4, 2024

(54) PREDICTION SYSTEM, METHOD AND COMPUTER READABLE MEDIUM

(71) Applicant: TOYOTA JIDOSHA KABUSHIKI KAISHA, Toyota-shi (JP)

(72) Inventors: Nagisa TANAKA, Toyota-shi (JP); Noriaki SAKAI, Nisshin-shi (JP); Hiroshi OSADA, Nagakute-shi (JP); Mitsuyoshi KAWAKAMI, Nagakute-shi (JP); Keisuke ICHIGE, Nagakute-shi (JP)

(73) Assignee: TOYOTA JIDOSHA KABUSHIKI KAISHA, Toyota-shi (JP)

(21) Appl. No.: 18/214,713

(22) Filed: Jun. 27, 2023

(30) Foreign Application Priority Data
Jun. 30, 2022 (JP) 2022-106484

(51) Int. Cl. A61B 5/11; G16H 20/30; A61B 5/00

(52) U.S. Cl. CPC A61B 5/1116; A61B 2503/20; A61B 5/7275; G16H 20/30

(57) ABSTRACT

A method executed by a computer according to one embodiment includes: extracting a duration time of one or more physical states of a posture or a motion that a person assumes or performs during a performing of a task by analyzing a process of the task of the person; acquiring a load on a body of the person regarding the one or more physical states; and predicting at least one of a fatigue level or a residual physical strength of the person in the task based on the extracted duration time of the one or more physical states and the acquired load.

### CROSS REFERENCE TO RELATED APPLICATIONS

This application is based upon and claims the benefit of priority from Japanese patent application No, 2022-106484, filed on Jun. 30, 2022, the disclosure of which is incorporated herein in its entirety by reference.

### BACKGROUND

The present disclosure relates to a prediction system, a method and a computer readable medium.

When human beings continue physical activities for some time, fatigue (muscle fatigue) occurs in their muscles involved in these activities. This can lead to a decrease in performance of activities and a decrease in work efficiency, etc. As of the present time, various studies for analyzing the phenomenon of muscle fatigue have been conducted.

For example, Ting Xia, Laura A. Frey Law, “A theoretical approach for modeling peripheral muscle fatigue and recovery”, Journal of Biomechanics, 41 (2008), pp. 3046-3052 defines a state transition model regarding fatigue, activation, and standby of muscles based on a motor unit (movement unit).

### SUMMARY

When a business operator considers or improves working plan at a construction site or the like, it is important for a company both from the point of view of the worker's health and that of efficient planning to make a plan in which fatigue caused by tasks performed by the workers is taken into account. However, the model described in Ting Xia, Laura A. Frey Law, “A theoretical approach for modeling peripheral muscle fatigue and recovery”, Journal of Biomechanics, 41 (2008), pp. 3046-3052 merely defines a transition of a muscle state including fatigue, and specific contents of the tasks are not taken into account. Therefore, it is possible that the fatigue of the workers may not be accurately predicted.

The present disclosure has been made in order to solve the aforementioned problem and provides a prediction system capable of accurately predicting at least one of a fatigue level or a residual physical strength in a task.

A prediction system according to one illustrative aspect of the present disclosure includes: an extraction unit configured to extract a duration time of one or more physical states of a posture or a motion that a person assumes or performs during a performing of a task by analyzing a process of the task of the person; an acquisition unit configured to acquire a load on a body of the person regarding the one or more physical states; and a prediction unit configured to predict at least one of a fatigue level or a residual physical strength of the person in the task based on the duration time of the one or more physical states extracted by the extraction unit and the load acquired by the acquisition unit. Since the prediction in the prediction system is performed based on the load regarding a posture or a motion that a person assumes or performs during the performing of the task and the duration time, the duration time being extracted by analyzing the process of the task, whereby it is possible to execute prediction based on the specific content of the task. Accordingly, it becomes possible to accurately predict at least one of a fatigue level or a residual physical strength in a task. Note that this prediction system is able to further improve the accuracy of the prediction by, for example, a method of machine learning (e.g., a method of updating a learning model by a system).

According to the present disclosure, it is possible to provide a prediction system, a method and a computer readable medium capable of accurately predicting at least one of a fatigue level or a residual physical strength in a task.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing one example of a prediction system according to a first embodiment;

FIG. 2 is a schematic view showing one example of a fatigue model according to the first embodiment;

FIG. 3A shows one example of a posture during a performing of a task according to the first embodiment;

FIG. 3B shows one example of a posture during the performing of the task according to the first embodiment;

FIG. 3C shows one example of a posture during the performing of the task according to the first embodiment;

FIG. 4 is an example of a graph showing transitions of a fatigue level of a worker according to the first embodiment;

FIG. 5 is an example of a graph showing transitions of a residual physical strength of the worker according to the first embodiment;

FIG. 6 is a flowchart showing a schematic processing example executed by a recovery prediction system according to the first embodiment; and

FIG. 7 is a block diagram showing one example of a hardware configuration of a prediction system.

### DESCRIPTION OF EMBODIMENTS

### First Embodiment

Hereinafter, with reference to the drawings, embodiments of the present disclosure will be described. The following descriptions and the drawings are omitted and simplified as appropriate for the sake of clarity of the description.

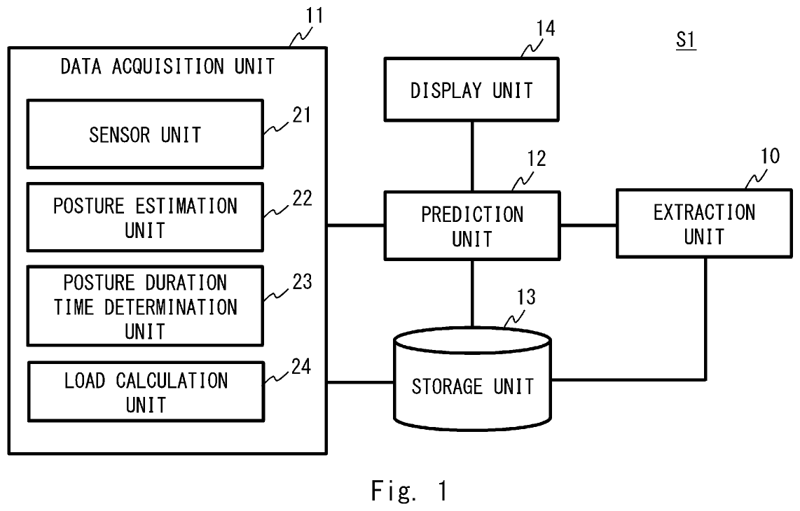

FIG. 1 is a diagram for describing a prediction system according to an embodiment. As shown in FIG. 1, a prediction system S1 according to this embodiment includes an extraction unit 10, a data acquisition unit 11, a prediction unit 12, a storage unit 13, and a display unit 14. Hereinafter, each part of the prediction system S1 will be described.

The extraction unit 10 analyzes one working process of a person, and extracts at least a duration time of one or more physical states of a posture or a motion that the person assumes or performs during a performing of the task. The working process to be analyzed is stored in the storage unit 13 as log data and the extraction unit 10 executes analysis and extraction processing using the log data. Further, the extraction unit 10 may extract a duration time of one or more physical states during the performing of the task and a timing when this duration occurs by analyzing the working process.

The term “posture” means a body shape assumed by a person and the term “motion” means that the person assumes a plurality of series of postures. Examples of the motion may include, for example, carrying a load, drilling holes in a column, receiving and passing jigs, or removing nails. The task to be analyzed may include a plurality of sections during which one or more physical states continue. This corresponds to a case in which a person assumes one posture for a predetermined time or longer, temporarily stops assuming this posture, and then continues the same posture again for a predetermined time or longer or a case in which a subject continues to assume a posture for a predetermined time or longer, and then continues to assume a different posture for a predetermined time or longer.

The data acquisition unit 11 acquires data of a load of one person regarding one or more physical states which this person is in during the performing of the task. The one or more physical states to be acquired are the same as the physical states to be extracted by the extraction unit 10. Specifically, the data acquisition unit 11 includes a sensor unit 21, a posture estimation unit 22, a posture duration time determination unit 23, and a load calculation unit 24.

The sensor unit 21 includes a plurality of inertial sensors attached to respective parts of the subject's body (e.g., at least one of parts such as an upper limb, a lower limb, the body trunk, or the head). Each of the inertial sensors acquires data regarding an inertial movement of a respective part when the subject assumes a posture regarding which the data of the load is to be acquired (posture that the subject assumes during the performing of the task). The data continues to be acquired from the sensor unit 21 until the task of the subject is ended.

The posture estimation unit 22 integrates data of the inertial sensors obtained from the sensor unit 21 and analyzes the integrated data to estimate the position and the direction of each part of the subject's body at each time during a performing of the task, i.e., the postures of the body. In other words, data of each inertial sensor is converted into posture data by the posture estimation unit 22. Further, the posture duration time determination unit 23 determines, for each posture, how long one or more postures estimated by the posture estimation unit 22 have been continued within a time of performance of the task to be measured.

The load calculation unit 24 specifies one or more postures regarding which the posture duration time determination unit 23 has determined that its duration time in the measurement is a predetermined threshold time or larger or its duration time with respect to a measurement time is a predetermined ratio or larger. The load calculation unit 24 then calculates a muscle load applied to each part of the subject's body in one or more specified postures by applying the posture data to a calculation model stored in the storage unit 13 in advance. The calculation model calculates the muscle load at each part by calculating data of the magnitude and the direction of an external force acting on each part (e.g., each joint) of the body in the specified posture. While the muscle load of each part is calculated, for example, as a numerical value of % MVC, which is a ratio with respect to Maximum Voluntary Contraction (MVC), it may be calculated as a numerical value of another type. Further, this muscle load may vary depending on the time. The muscle load of each posture is calculated as described above.

Note that the sensor unit 21 may include a plurality of sensors such as displacement sensors or bending sensors instead of the inertial sensors. In this case as well, the posture estimation unit 22 is able to estimate the posture that the subject assumes based on the data obtained from the sensor unit 21.

Further, instead of the sensor unit 21, a camera that captures the subject's posture by a still image or a moving image may be provided. The posture estimation unit 22 estimates the posture that the subject assumes by analyzing the image captured by the camera using an image recognition technique. The posture estimation unit 22 may determine the subject's posture by a method of machine learning or the system may update the model of posture estimation. In this way, any existing motion capture technique may be used for measurement of data, regarding the subject's posture in the data acquisition unit 11 and estimation of the posture based on the acquired data.

Further, the data acquisition unit 11 may presuppose the subject's posture using a computer simulation, and calculate the load data in this posture instead of calculating load data by acquiring the actual data regarding the subject's posture as described above. Further, the data acquisition unit 11 may be an interface that simply acquires data of the load regarding the physical state measured or predicted by an apparatus other than the prediction system S1 from the other apparatus.

Next, the prediction unit 12 will be described. The prediction unit 12 acquires data of the duration time of the one or more physical states, the data being extracted by the extraction unit 10, and data of the muscle load of one or more postures at each part of the subject, the data being calculated by the load calculation unit 24. Then, the prediction unit 12 performs computation of accumulating, regarding each posture, the fatigue level in this posture in a time during which this posture is kept. Accordingly, the prediction unit 12 predicts the fatigue level of the person during the performing of the task in which one or more postures are continuously performed. The predicted fatigue level is a value at one or more desired timings from a start of a task to an end of the task. The predicted value may include, for example, the value of the fatigue level when the task is ended or may include the value of the fatigue level at one or more timings before the end of the task. As will be described later, the prediction unit 12 is able to successively predict fatigue levels from the start of the task to the end of the task.

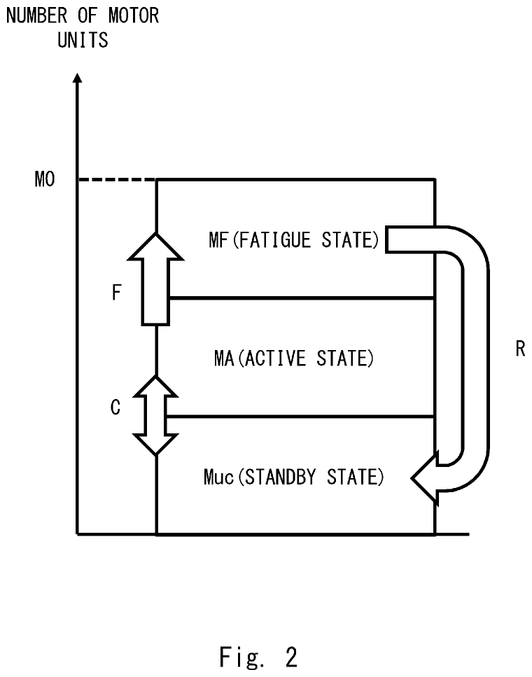

Referring to FIG. 2, an example of the model that the prediction unit 12 uses to predict the fatigue level of a person will be described. The model explained in this example is a model described in Ting Xia, Laura A. Frey Law, “A theoretical approach for modeling peripheral muscle fatigue and recovery”, Journal of Biomechanics, 41 (2008), pp. 3046-3052, which is stored in the storage unit 13 in advance. In this model, it is assumed that there is a predetermined motor unit (movement unit) in a muscle at a part of the body and the state transition at the time of each motor unit is calculated, whereby it is possible to determine the state of the muscle fatigue at a specified part. It is assumed that each of the motor units is in any one of a standby state, an active state, and a fatigue state, the number of motor units that are in the standby state is denoted by Muc, the number of motor units that are in the active state is denoted by MA, and the number of motor units that are in the fatigue state is denoted by MF. Then, when the total number of motor units is denoted by MO (=Muc+MA+MF), a ratio of the number of motor units that are in the standby state or the active state ((Muc+MA)/MO) is defined to be a residual physical strength and a ratio of the number of motor units that are in the fatigue state (MF/MO) is defined to be a fatigue level.

Further, each motor unit may make a transition from the standby state to the active state, from the active state to the standby state, a transition from the active state to the fatigue state, and a transition from the fatigue state to the standby state. Regarding the degree of the transition from the standby state to the active state or the degree of the transition from the active state to the standby state, an active strength parameter C is defined. Regarding the degree of the transition from the active state to the fatigue state, a fatigue strength parameter F is defined. Regarding the degree of the transition from the fatigue state to the standby state, a recovery strength parameter R is defined. These parameters define the processes of activation, fatigue, and recovery of the muscle.

ftp this model, the following expressions are defined as changes of Muc, MA, and MF over time.

$$[ Expression 1 ]$$

$$\frac{dMuc}{dt} = - C ( t ) + R \cdot MF \qquad ( 1 )$$

$$[ Expression 2 ]$$

$$\frac{dMA}{dt} = C ( t ) - F \cdot MA \qquad ( 2 )$$

$$[ Expression 3 ]$$

$$\frac{dMF}{dt} = F \cdot MA - R \cdot MF \qquad ( 3 )$$

The active strength parameter C(t) is a variable at time t (the value in accordance with the muscle load at time t), and the prediction unit 12 is able to determine C in accordance with the change in the posture estimated by the posture estimation unit 22 over time. The symbol C(t) is defined as follows in accordance with the value of the parameter % MVC (command value indicating the muscle load applied to each part of the body).

$$[ Expression 4 ]$$

$$( 4 )$$

$$C ( t ) = \{ \begin{matrix}LD \cdot ( \% MVC - MA ) & If MA < \% MVC and MA + Muc \geq \% MVC \\ LD \cdot Muc & If MA < \% MVC and MA + Muc < \% MVC \\ LR \cdot ( \% MVC - MA ) & If MA \geq \% MVC\end{matrix}$$

The symbols LD and LR in (4) are predetermined coefficients. When % WC (command value) is small (a value close to 0), the recovery strength parameter R may be multiplied by the predetermined coefficient r in view of intramuscular vasodilation. The details thereof are described in John M. Looft, Nicole Herkert, Laura Frey-Law, “Modification of a three-compartment muscle fatigue model to predict peak torque decline during intermittent tasks”, Journal of Biomechanics, 77 (2018), pp. 16-25.

The prediction unit 12 applies the muscle load of each part in the specified posture and the duration time of the posture during the performing of the task as parameters to Expressions (1)-(4) of the model shown above. Accordingly, the prediction unit 12 calculates at least one of ME or Muc+MA at the timing when the task is ended. When there are a plurality of sections during which a specified posture continues during the performing of the task, the prediction unit 12 is able to calculate MF accumulated for the duration time of the posture for each section and accumulate the calculated fatigue level for all the sections, thereby calculating MF when the task is ended. Accordingly, the prediction unit 12 is able to predict at least one of the fatigue level (MF/MO) or the residual physical strength ((Muc+MA)/MO) of the subject at the timing when the task is ended. However, the prediction method is not limited to the above specific example.

As described above, the storage unit 13 stores log data of the working process used by the extraction unit 10, and the models used by the data acquisition unit 11 and the prediction unit 12. The display unit 14 is an interface such as a display that displays the result of the prediction calculated by the prediction unit 12 and presents this result to the user. The display unit 14 may include a speaker or the like as an interface for sending the result of the prediction or some kind of other notification or proposal to the user.

Next, examples of results of the prediction calculated by the prediction unit 12 will be shown.

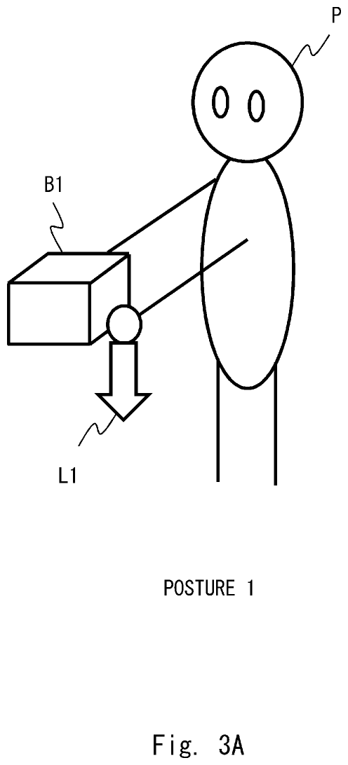

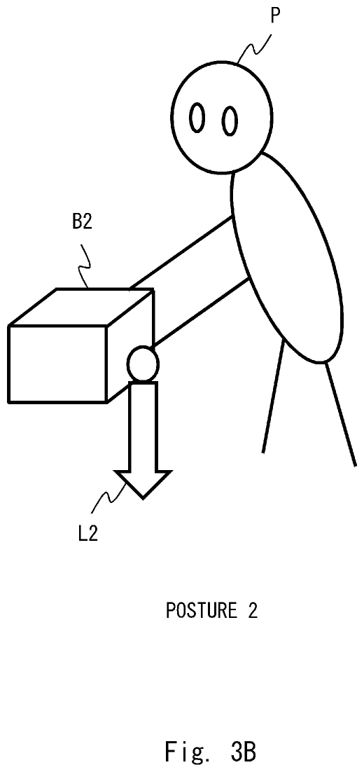

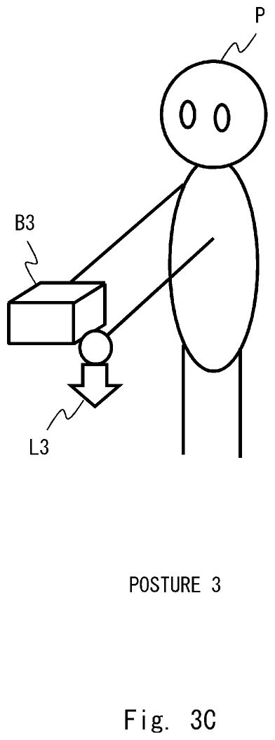

FIGS. 3A-3C show examples of a plurality of postures continued for a predetermined threshold time or longer in the task to be measured. A posture 1 shown in FIG. 3A shows a state in which a person P holds a load B1. Further, a posture 2 shown in FIG. 3B shows a state in which the person P holds a load B2 and a posture 3 shown in FIG. 3C shows a state in which the person P holds a load B3. In this case, the weight of the load is heavier in the order of the loads B2, B1, and B3. Therefore, a muscle load. L applied to a specific part of the subject (e.g., an arm or the hip) is greater in the order L2 shown in FIG. 3B, L1 shown in FIG. 3A, and L3 shown in FIG. 3C.

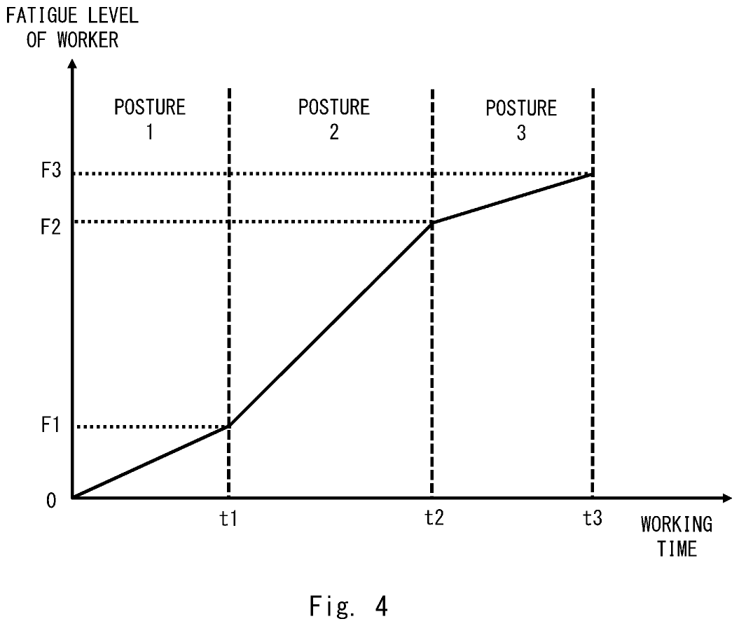

FIG. 4 is an example of a graph showing transitions of the fatigue level (MF/MO) [%] of the worker (subject) calculated by the prediction unit 12 in a state in which the posture 1 continues for a period t1, the posture 2 continues for a period (t2−t1), and the posture 3 continues for a period (t3−t2) in a task. While the fatigue level is 0 at time 0, the fatigue level is F1 at time t1 when the posture 1 is ended, the fatigue level is F2 at time t2 when the posture 2 is ended, and the fatigue level is F3 at time t3 when the posture 3 is ended. As described above, the muscle load L is greater in the order L2, L1, and L3. Therefore, the degree of the increase in the fatigue level is greater in the order of the posture 2, the posture 1, and the posture 3.

The extraction unit 10 extracts each of the duration time of the posture 1, that of the posture 2, and that of the posture 3 during the performing of the tasks by analyzing the predetermined working processes. The data acquisition unit 11 acquires L1-L3, which are muscle loads L of the respective postures 1-3. The prediction unit 12 predicts, based on the duration time of each posture that has been extracted and the muscle loads L1-L3, the fatigue level of the person during the performing of each task and at the timing when each task is ended, as shown in the graph in FIG. 4. The prediction unit 12 causes the display unit 14 to display the calculated graph. Accordingly, the user is able to know transitions of the fatigue level during the performing of the task and the fatigue level after the task is ended.

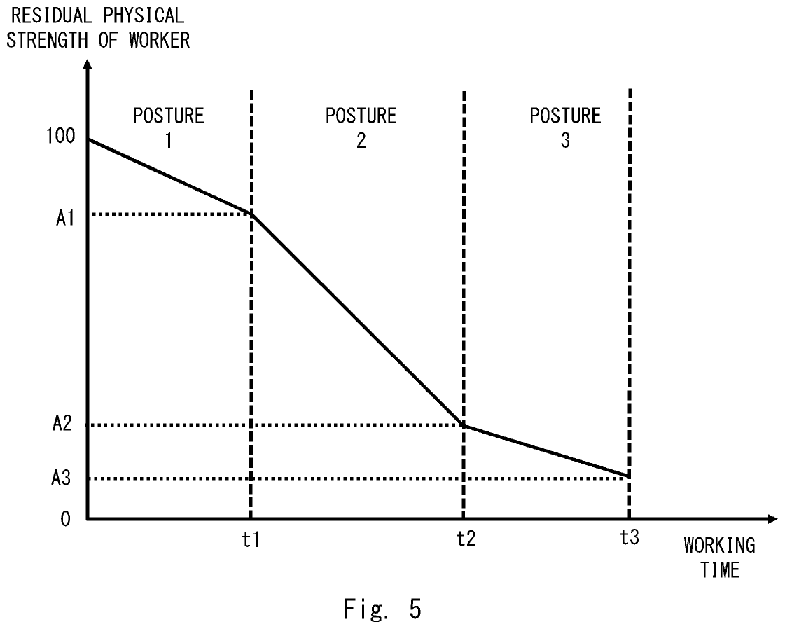

FIG. 5 is an example of a graph showing transitions of the residual physical strength ((Muc+MA)/MO) [%] of the worker calculated by the prediction unit 12 in a situation similar to that in FIG. 4. While the residual physical strength is 100 at time 0, the residual physical strength is A1 (=100−F1) at time t1 when the posture 1 is ended, the residual physical strength is A2 (=100−F2) at time t2 when the posture 2 is ended, and the residual physical strength is A3 (=100−F3) at time t3 when the posture 3 is ended. In this manner, the prediction unit 12 may calculate the transition state of the residual physical strength and present the calculated transition state to the user by the display unit 14.

In the example described above, the prediction unit 12 predicts the fatigue level or the residual physical strength during the performing of the task and at the timing when the task is ended using the muscle load of each part regarding one or more postures and the duration time of each posture during the performing of the task. However, it is possible to predict, regarding a motion including a plurality of postures as well, the fatigue level or the residual physical strength during the performing of the task and at the timing when the task is ended using the muscle load of each part in one or more motions and the duration time of each motion during the performing of the task. Further, prediction similar to that stated above may be performed also in a case in which both a section during which a posture continues and a section during which a motion continues are included during the performing of the task. Since details of the prediction processing are similar to those stated above, the descriptions thereof will be omitted. Further, while there is a section in one task where the postures 1-3 continue in the examples shown in FIGS. 3-5, similar prediction processing may be executed also in a case in which there are a plurality of tasks, such as a task 1 in which the posture 1 continues, a task 2 in which the posture 2 continues, and a task 3 in which the posture 3 continues.

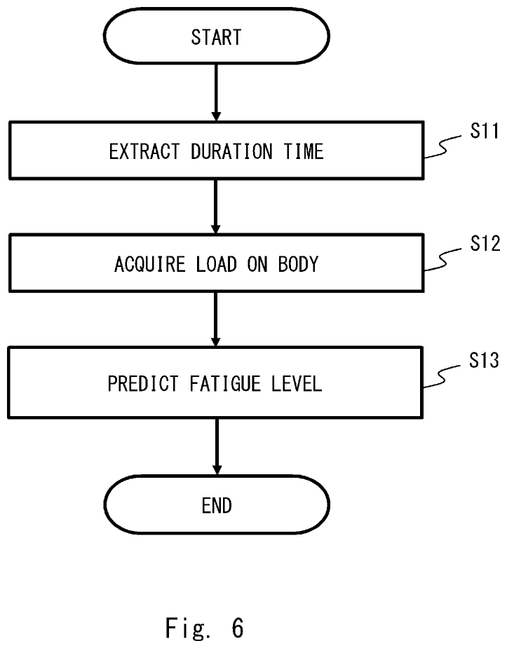

FIG. 6 is a flowchart showing a schematic processing example executed by the prediction system S1 and the executed processing will be described with reference to FIG. 6. First, the extraction unit 10 extracts the duration time of the one or more physical states of the posture or the motion that the person assumes or performs during the performing of the task by analyzing the process of the task of the person (Step S11) Further, the data acquisition unit 11 acquires data of the load on the person regarding one or more physical states which the person may be in during the performing of the task (Step S12), The processing that the sensor unit 21 to the load calculation unit 24, which are parts of the data acquisition unit 11, executes in order to acquire the data of the load has been described above, and the descriptions thereof will be omitted. One of the processing of Step S11 and the processing of Step S12 may be executed first or they may be executed in parallel to each other.

The prediction unit 12 predicts a fatigue level of the person in a task based on the duration time of one or more physical states extracted in Step S11 and the load acquired in Step S12 (Step S13). Alternatively, in Step S13, the prediction unit 12 may predict the residual physical strength of the person in place of or in addition to the fatigue level of the person.

As described above, the prediction in the prediction system S1 is performed based on the load regarding the posture or the motion that the person assumes or performs and a duration time of the task extracted by analyzing the working process. Therefore, it is possible to execute the prediction based on the specific content of the task. Therefore, it is possible to accurately predict at least one of a fatigue level or a residual physical strength in a task. For example, the present disclosure is useful for predicting the fatigue level at a construction site or the like where the content of the task may change.

Even in a case in which a task includes a plurality of sections during which one or more physical states continue, the prediction unit 12 is able to predict at least one of the fatigue level or the residual physical strength at the timing when the task is ended by accumulating the fatigue levels calculated in the respective sections. Therefore, even when a task includes various postures or motions, it becomes possible to accurately predict a fatigue level or a residual physical strength.

Further, the present disclosure may have the following variations.

For example, the storage unit 13 may further store, as data of the working process to be analyzed, data of a working environment of the task. The working environment changes (with time) as the task performed by the worker proceeds in a construction or the like. Therefore, even when a task including one or more same physical states (postures or motions) is performed, a duration time of the physical state during the performing of the task may change with time. In this case, the extraction unit 10 recognizes that the working environment changes with time by referring to data of the working environment stored in the storage unit 13, detects that the duration time of one or more physical states for each section changes with time, and extracts a duration time for each section. While the extraction unit 10 is able to detect the change in the duration time by executing, for example, a simulation of the task, the detection method is not limited thereto. Then, the prediction unit 12 is able to predict at least one of a fatigue level or a residual physical strength at the timing when the task is ended based on the duration time for each section detected by the extraction unit 10 and data of the load acquired by the data acquisition unit 11, Note that the fatigue level or the residual physical strength during the performing of the task may be predicted as well. Accordingly, even when the working time is changed depending on the state of progress of the construction, the prediction system S1 is able to accurately perform prediction in accordance with the change in the working time.

Further, the prediction unit 12 may use not only a duration time of one or more physical states during the performing of the task but also information on the timing when the continuation of the physical state occurs, the information being extracted by the extraction unit 10, based on the self-evident fact that the solutions of the simultaneous differential equations (1)-(3) depend on the initial values of Muc, MA, and MF. By using these information items, the prediction unit 12 is able to predict at least one of a fatigue level or a residual physical strength in a task by executing computation of accumulating the fatigue level regarding each posture in a time during which this posture is kept, Depending oii the content of the task, even in a case in which the same posture is assumed for the same period of time, it is possible that the fatigue level caused by the continuation of the posture may differ depending on the timing when this posture is assumed. Even in this case, however, the prediction system S1 is able to perform accurate prediction.

Further, when the predicted value of the fatigue level at the timing when the task is ended or during the performing of the task is equal to or larger than a predetermined threshold, or when the value of the residual physical strength at the timing when the task is ended or during the performing of the task is smaller than a predetermined threshold, the prediction unit 12 may propose at least one of reduction in the working time to be predicted or a change in the content of the work. This proposal is presented to the user via, for example, the display unit 14. Accordingly, the user is able to change the working schedule so as to reduce the burden on the worker, whereby it is possible to mitigate the burden on the worker.

Further, the prediction unit 12 is also able to predict the burden that the worker subjectively feels. The prediction unit 12 uses data of a task other than the task to be predicted, the data being stored in the storage unit 13. The data of the other task includes a result of the prediction of at least one of a fatigue level or a residual physical strength of the person in the other task, and at least one of a subjective fatigue level or a subjective residual physical strength that the person has subjectively felt in the other task. The prediction of the fatigue level or the residual physical strength in the other task can be achieved by a method similar to that in the prediction regarding the task to be predicted shown above. This prediction may be performed by the prediction unit 12 or a prediction system other than the prediction system S1.

The subjective fatigue level or the subjective residual physical strength, which is subjective information indicating the fatigue level or the residual physical strength of the worker responded by the worker at a timing in the task (e.g., just after the task is ended), may be quantitative (numerical) information or qualitative information. Even when the content of the task is the same, the subjective fatigue level or the subjective residual physical strength responded by a worker may be different from the subjective fatigue level or the subjective residual physical strength responded by another worker. As one example of the quantitative information, the fatigue level (or the residual physical strength) may be indicated by values from 5 to 1 in a descending order of the fatigue level. As one example of the qualitative information, the fatigue level (or the residual physical strength) may be indicated by the magnitude thereof, such as “tired”, “rather tired”, “not very tired”, and “not tired”. However, the quantitative or qualitative information that can be used is not limited thereto.

The result of the prediction of the fatigue level or the residual physical strength and the subjective fatigue level or the subjective residual physical strength that corresponds to the result of the prediction, both of which being included in the data of the other task, are the values at the same (or corresponding) timing in the other task such as a timing just after the other task is ended. This timing may include one or more arbitrary timings in the other task. Further, the data of the other task may store data including the result of the prediction of each task and at least one of the subjective fatigue level or the subjective residual physical strength regarding a plurality of tasks, not one task.

The prediction unit 12 further predicts at least one of a subjective fatigue level or a subjective residual physical strength that the worker subjectively feels in a task by referring to the result of predicting at least one of a fatigue level or a residual physical strength of a person during the performing of the task and the data of the other task that is stored. The prediction unit 12 may execute this prediction using a predetermined algorithm or using a model using A1.

When at least one of two conditions, that is, a condition that the predicted quantitative and subjective fatigue level is equal to or larger than a predetermined threshold or a condition that the predicted quantitative and subjective residual physical strength is smaller than a predetermined threshold, is satisfied, the prediction unit 12 may send a notification to the user via, for example, the display unit 14 or may propose at least one of reduction in the working time or a change in the content of the task. Even in a case in which the predicted qualitative and subjective fatigue level or subjective residual physical strength indicates specific information, the prediction unit 12 is able to send a similar notification or make a similar proposal to the user. When, for example, the predicted quantitative and subjective fatigue level is “5” or “4” or when the predicted qualitative and subjective fatigue level is “tired” or “rather tired”, the prediction unit 12 is able to send the aforementioned notification or make the aforementioned proposal to the user.

Further, even in a case in which the worker actually executes the task and the fatigue level or the residual physical strength of this worker is acquired in real time, the prediction unit 12 may refer to the acquired value and data of the other task, thereby predicting a subjective fatigue level or a subjective residual physical strength in the task in real time. The data acquisition unit 11 and the prediction unit 12 acquire task data of the worker, thereby being able to acquire a duration time of one or more physical states of the physical postures or motions so far and its load, and derive at least one of the fatigue level or the residual physical strength in real time using the acquired data. Alternatively, real-time data of the fatigue level or the residual physical strength may be acquired by another device (e.g., wearable device attached to the worker). When at least one of the following three conditions, that is, the predicted quantitative and subjective fatigue level is equal to or larger than the predetermined threshold, the predicted quantitative and subjective residual physical strength is smaller than the predetermined threshold, or the predicted qualitative and subjective fatigue level or subjective residual physical strength indicate specific information, is satisfied, the prediction unit 12 detects this state. Upon detecting this state, the prediction unit 12 is able to send a notification indicating that the burden on the worker is too large. Alternatively, the prediction unit 12 is able to make a proposal to mitigate the burden on the worker such as the cancellation of the task that is currently being executed, or a change in the task.

Further, the prediction unit 12 is able to predict the fatigue level or the residual physical strength of not only one part of the subject's body but also those of a plurality of parts of the subject's body. The details of this prediction method have been described above. Accordingly, it is possible to visually recognize which part of the body of the worker is likely to or unlikely to fatigue, whereby it becomes possible to make a working plan that is a highly efficient or in which the total burden on the worker is small. At this time, when the predicted value of the fatigue level at the timing when the task is ended is equal to or larger than a predetermined threshold or the value of the residual physical strength at the time when the task is ended is smaller than a predetermined threshold in at least one part, the prediction unit 12 may propose at least one of reduction in the working time or a change in the content of the task.

Further, the prediction unit 12 may predict, regarding each of the plurality of parts, the burden that the worker subjectively feels, in a way similar to that described above. That is, the prediction unit 12 predicts at least one of a subjective fatigue level or a subjective residual physical strength that the worker subjectively feels regarding each part during the performing of the task by referring, regarding each of the parts, to the result of predicting at least one of a fatigue level or a residual physical strength of a person during the performing of the task and the data of the other task that is stored. The details of the prediction processing at each part have been described above. Further, when at least one of the following three conditions is satisfied at any part, that is, a condition that the predicted quantitative and subjective fatigue level is equal to or larger than a predetermined threshold, a condition that the predicted quantitative and subjective residual physical strength is smaller than a predetermined threshold, or a condition that the predicted qualitative and subjective fatigue level or subjective residual physical strength indicates specific information, the prediction unit 12 is able to send the notification or make the proposal as described above.

Further, even in a case in which the worker actually executes the task and the fatigue level or the residual physical strength of this worker is acquired in real time, the prediction unit 12 predicts, for each part, at least one of a subjective fatigue level or a subjective residual physical strength that the worker actually and subjectively feels. Then, as described above, it is possible to send a notification or make a proposal for reducing the burden on the worker as necessary.

The model that the prediction unit 12 uses is not limited to the aforementioned one. Further, the user may input a feedback indicating a correct or incorrect result of the prediction displayed by the display unit 14 into the prediction system S1. The prediction system S1 is able to update the model used by the prediction unit 12 using A1 based on the feedback. Accordingly, it is possible to further improve the accuracy of predicting at least one of a fatigue level or a residual physical strength predicted by the prediction unit 12, or at least one of a subjective fatigue level or a subjective residual physical strength.

In the aforementioned embodiments, the present disclosure has been described as a hardware configuration. However, the present disclosure may execute the processing (step) of the prediction system S1 described in the aforementioned embodiments by causing a processor in the computer to execute a computer program.

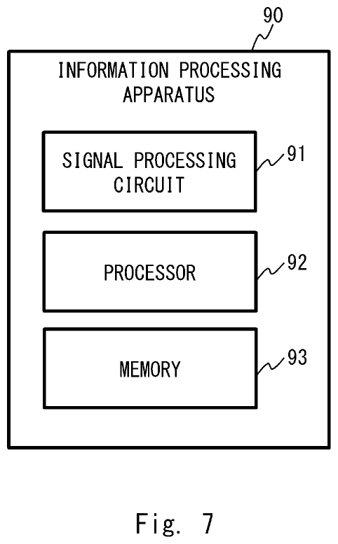

FIG. 7 is a block diagram showing a hardware configuration example of an information processing apparatus in which processing of each embodiment described above is executed. Referring to FIG. 7, this information processing apparatus 90 includes a signal processing circuit 91, a processor 92, and a memory 93.

This signal processing circuit 91 is a circuit for processing signals in accordance with the control of the processor 92. The signal processing circuit 91 may include a communication circuit that performs at least one of receiving a signal from a transmission apparatus or transmitting a signal to a receiving apparatus.

The processor 92 is connected to (coupled to) the memory 93, and loads software (computer program) from the memory 93 and executes the loaded software (computer program), thereby performing processing of the apparatus described in the aforementioned embodiments. As one example of the processor 92, one of a Central Processing Unit (CPU), a Micro Processing Unit (MPU), a Field-Programmable Gate Array (FPGA), a Demand-Side Platform (DSP), and an Application Specific Integrated Circuit (ASIC) may be used or some of them may be used in parallel.

The memory 93 is composed of a volatile memory, a non-volatile memory, or a combination of them. The number of memories 93 is not limited to one and a plurality of memories 93 may be provided. The volatile memory may be, for example; a Random Access Memory (RAM) such as a Dynamic Random Access Memory (DRAM) or a Static Random Access Memory (SRAM). The non-volatile memory may be, for example, a Read Only Memory (ROM) such as a Programmable Random Only Memory (PROM) or an Erasable Programmable Read Only Memory (EPROM), a flash memory, or a Solid State Drive (SSD).

The memory 93 is used to store one or more instructions. One or more instructions are stored in the memory 93 as software modules. The processor 92 loads these software modules from the memory 93 and executes these loaded software modules, thereby performing processing described in the aforementioned embodiments. The memory 93 may be provided in a desired place.

As described above, one or more processors that each apparatus has according to the aforementioned embodiments executes one or more programs including instructions for causing a computer to execute the algorithm described with reference to the drawings. According to the aforementioned processing, the fatigue level prediction method described in each embodiment can be achieved.

The program includes instructions (or software codes) that, when loaded into a computer, cause the computer to perform one or more of the functions described in the embodiments. The program may be stored in a non-transitory computer readable medium or a tangible storage medium. By way of example, and not a limitation, computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (registered trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

The whole or part of the embodiments disclosed above can be described as, but not limited to, the following supplementary notes.

### (Supplementary Note 1)

A Prediction System Comprising:
an extraction unit configured to extract a duration time of one or more physical states of a posture or a motion that a person assumes or performs during a performing of a task by analyzing a process of the task of the person;
an acquisition unit configured to acquire a load on a body of the person regarding the one or more physical states; and
a prediction unit configured to predict at least one of a fatigue level or a residual physical strength of the person in the task based on the duration time of the one or more physical states extracted by the extraction unit and the load acquired by the acquisition unit.

### (Supplementary Note 2)

The prediction system according to Supplementary Note 1, wherein the task includes a plurality of sections during which the one or more physical states continue, and
the prediction unit predicts at least one of a fatigue level or a residual physical strength of the person when the task is ended by calculating a fatigue level caused by the load for each of the sections and accumulating the calculated fatigue levels in all the sections.

### (Supplementary Note 3)

The prediction system according to Supplementary Note 2, wherein the extraction unit detects that the duration time of the one or more physical states for each of the sections is changed with time based on an environment in which the task is performed being changed with time, and the prediction unit predicts at least one of a fatigue level or a residual physical strength of the person when the task is ended based on the duration time for each of the sections detected by the extraction unit and the load acquired by the acquisition unit.

### (Supplementary Note 4)

The prediction system according to any one of Supplementary Notes 1 to 3, wherein
the extraction unit extracts the duration time of the one or more physical states during the performing of the task and a timing when this duration occurs by analyzing the process of the task, and
the prediction unit predicts at least one of a fatigue level or a residual physical strength of the person in the task based on the duration time and the timing extracted by the extraction unit and the load acquired by the acquisition unit.

### (Supplementary Note 5)

The prediction system according to any one of Supplementary Notes 1 to 4, wherein
the acquisition unit acquires loads at a plurality of parts of the body of the person regarding the one or more physical states, and
the prediction unit predicts at least one of fatigue levels or residual physical strengths at the plurality of parts of the person in the task based on the duration time of the one or more physical states extracted by the extraction unit and loads at the plurality of parts acquired by the acquisition unit.

### (Supplementary Note 6)

The prediction system according to any one of Supplementary Notes 1 to wherein the prediction unit further predicts at least one of a subjective fatigue level or a subjective residual physical strength that the person subjectively feels in the task by referring to data of another task including a result of predicting at least one of a fatigue level or a residual physical strength of a person in the other task and at least one of a subjective fatigue level or a subjective residual physical strength that the person has subjectively felt in the other task, and a result of predicting at least one of a fatigue level or a residual physical strength of the person in the task.

### (Supplementary Note 7)

The prediction system according to any one of Supplementary Notes 1 to 6, wherein the prediction unit proposes at least one of reduction in the working time or a change in the content of the task when the value of the fatigue level that has been predicted is equal to or larger than a predetermined threshold or the value of the residual physical strength is smaller than a predetermined threshold.

### (Supplementary Note 8)

The prediction system according to Supplementary Note 5, wherein the prediction unit proposes at least one of reduction in the working time or a change in the content of the task when the value of the fatigue level that has been predicted in at least one of the parts is equal to or larger than a predetermined threshold or the value of the residual physical strength is smaller than a predetermined threshold.

### (Supplementary Note 9)

A Method Executed by a Computer, the Method Comprising:
extracting a duration time of one or more physical states of a posture or a motion that a person assumes or performs during a performing of a task by analyzing a process of the task of the person;
acquiring a load on a body of the person regarding the one or more physical states; and
predicting at least one of a fatigue level or a residual physical strength of the person in the task based on the extracted duration time of the one or more physical states and the acquired load.

### (Supplementary Note 10)

A non-transitory computer readable medium storing a program that causes a computer to execute:
extracting a duration time of one or more physical states of a posture or a motion that a person assumes or performs during a performing of a task by analyzing a process of the task of the person;
acquiring a load on a body of the person regarding the one or more physical states; and
predicting at least one of a fatigue level or a residual physical strength of the person in the task based on the extracted duration time of the one or more physical states and the acquired load.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A prediction system comprising:
an extraction unit configured to extract a duration time of one or more physical states of a posture or a motion that a person assumes or performs during a performing of a task by analyzing a process of the task of the person;
an acquisition unit configured to acquire a load on a body of the person regarding the one or more physical states; and
a prediction unit configured to predict at least one of a fatigue level or a residual physical strength of the person in the task based on the duration time of the one or more physical states extracted by the extraction unit and the load acquired by the acquisition unit.

2. The prediction system according to claim 1, wherein
the task includes a plurality of sections during which the one or more physical states continue, and
the prediction unit predicts at least one of a fatigue level or a residual physical strength of the person when the task is ended by calculating a fatigue level caused by the load for each of the sections and accumulating the calculated fatigue levels in all the sections.

3. The prediction system according to claim 2, wherein
the extraction unit detects that the duration time of the one or more physical states for each of the sections is changed with time based on an environment in which the task is performed being changed with time, and
the prediction unit predicts at least one of a fatigue level or a residual physical strength of the person when the task is ended based on the duration time for each of the sections detected by the extraction unit and the load acquired by the acquisition unit.

4. The prediction system according to claim 1, wherein
the extraction unit extracts the duration time of the one or more physical states during the performing of the task and a timing when this duration occurs by analyzing the process of the task, and
the prediction unit predicts at least one of a fatigue level or a residual physical strength of the person in the task based on the duration time and the timing extracted by the extraction unit and the load acquired by the acquisition unit.

5. The prediction system according to claim 1, wherein
the acquisition unit acquires loads at a plurality of parts of the body of the person regarding the one or more physical states, and
the prediction unit predicts at least one of fatigue levels or residual physical strengths at the plurality of parts of the person in the task based on the duration time of the one or more physical states extracted by the extraction unit and loads at the plurality of parts acquired by the acquisition unit.

6. The prediction system according to claim 1, wherein the prediction unit further predicts at least one of a subjective fatigue level or a subjective residual physical strength that the person subjectively feels in the task by referring to data of another task including a result of predicting at least one of a fatigue level or a residual physical strength of a person in the other task and at least one of a subjective fatigue level or a subjective residual physical strength that the person has subjectively felt in the other task, and a result of predicting at least one of a fatigue level or a residual physical strength of the person in the task.

7. The prediction system according to claim 1, wherein the prediction un proposes at least one of reduction in the working time or a change in the content of the task when the value of the fatigue level that has been predicted is equal to or larger than a predetermined threshold or the value of the residual physical strength is smaller than a predetermined threshold.

8. The prediction system according to claim 5, wherein the prediction unit proposes at least one of reduction in the working time or a change in the content of the task when the value of the fatigue level that has been predicted in at least one of the parts is equal to or larger than a predetermined threshold or the value of the residual physical strength is smaller than a predetermined threshold.

9. A method executed by a computer, the method comprising:
extracting a duration time of one or more physical states of a posture or a motion that a person assumes or performs during a performing of a task by analyzing a process of the task of the person;
acquiring a load on a body of the person regarding the one or more physical states; and
predicting at least one of a fatigue level or a residual physical strength of the person in the task based on the extracted duration time of the one or more physical states and the acquired load.

10. A non-transitory computer readable medium storing a program that causes a computer to execute:
extracting a duration time of one or more physical states of a posture or a motion that a person assumes or performs during a performing of a task by analyzing a process of the task of the person;
acquiring a load on a body of the person regarding the one or more physical states; and
predicting at least one of a fatigue level or a residual physical strength of the person in the task based on the extracted duration time of the one or more physical states and the acquired load.
